(19) United States
(12) Patent Application Publication
(10) Pub. No.: US 2002/0007047 A1
(43) Pub. Date: Jan. 17, 2002

(54) Short forms of chemokine beta-8

(76) Inventors: John White, Coatsville, PA (US); Edward Appelbaum, Blue Bell, PA (US); Daniel O'Shannessy, Limerick, PA (US); James Allan Fornwald, Norristown, PA (US); Kevin O'Donnell, Amber, PA (US)

Correspondence Address: STERNE, KESSLER, GOLDSTEIN & FOX P.L.L.C., Suite 600, 1100 New York Avenue, N.W., Washington, DC, 20005-3934, US

(21) Appl. No.: 09/778,852

(22) Filed: Feb. 8, 2001

Related U.S. Application Data
(63) Continuation of application No. 09/338,510, filed on Jun. 23, 1999.
Application No. 09/338,510 is a continuation of application No. 09/043,811, filed on Oct. 21, 1998, now abandoned.
Application No. 09/043,811 is a 371 of international application No. PCT/US96/15592, filed on Sep. 27, 1996.
(60) Provisional application No. 60/004,517, filed on Sep. 29, 1995.

(51) Int. Cl. A61K 38/19; C12P 21/02; C12N 5/06; C07K 14/52

(52) U.S. Cl. 530/351; 424/85.1; 435/69.5; 435/325

(57) ABSTRACT

Novel polypeptides of the truncated form of human Ckβ-8 are provided. Methods for using the polypeptides are also provided.

### BACKGROUND OF THE INVENTION

This invention relates to polypeptide and polynucleotide sequences encoding a short form of Chemokine Beta-8 (Ckβ-8). In particular, the present invention relates to the use of the polynucleotide and polypeptide sequences, the production of the polynucleotide and polypeptide sequences, and inhibiting the action of the polypeptide. Chemokines, also referred to as intercrine cytokines, are a subfamily of structurally and functionally related cytokines. These molecules are small, inducible, proinflammatory, proteins. In general, chemokines exhibit 20% to 75% homology at the amino acid level and are characterized by four conserved cysteine residues that form two disulfide bonds. Based on the arrangement of the first two cysteine residues, chemokines have been classified into two subfamilies, alpha and beta. In the alpha subfamily, the first two cysteines are separated by one amino acid and are therefore referred to as the “C-X-C” subfamily. In the beta subfamily, the two cysteines are in an adjacent position and are thus referred to as the “C-C” subfamily. The first cysteine forms a disulfide bond with the third cysteine, and the second with the fourth, resulting in a similar tertiary structure for many of the chemokines. Structurally, many chemokines undergo proteolytic processing to yield a functional, “mature” protein. This usually involves cleavage of a short leader sequence on the N-terminal portion of the protein. At least 14 distinct α-chemokines and 12 β-chemokines have been described at the protein and/or cDNA level.

The intercrine cytokines exhibit a wide variety of functions. One important feature is their ability to stimulate chemotactic migration of distinct cell types such as monocytes, neutrophils, T lymphocytes, basophils, and fibroblasts. The α and β subfamilies differ somewhat in their cell target selectivity. Most of the α chemokines attract neutrophils, fibroblasts, T cells, and NK cells. β-chemokines primarily attract monocytes and T lymphocytes. Many chemokines have proinflammatory activity and are involved in multiple steps during an inflammatory reaction. These activities include stimulation of histamine release, lysosomal enzyme and leukotriene release, increased adherence of target immune cells to endothelial cells, enhanced binding of complement proteins, induced expression of granulocyte adhesion molecules and complement receptors, and respiratory burst. In addition to their involvement in inflammation, certain chemokines have been shown to exhibit other activities. For example, macrophage inflammatory protein (MIP-1) is able to suppress hematopoietic stem cell proliferation, platelet factor-4 (PF4) is a potent inhibitor of endothelial cell growth, Interleukin-8 (IL-8) promotes proliferation of keratinocytes, and GRO is an autocrine growth factor for melanoma cells. Chemokines have been implicated in a number of physiological and disease conditions, particularly those that have an inflammatory component. These include but are not limited to: lymphocyte trafficking, wound healing, hematopoietic regulation and immunological disorders such as allergy, asthma and arthritis. Many chemokines were initially isolated as products of an inflammatory response For example, MIP-1 was originally isolated as an endotoxin-induced proinflammatory cytokine produced by macrophages. Other members of this subfamily have similarly been identified, based on inflammation induction as well as amino acid sequence homology. This includes the full length as well as the shortened forms of the Ckβ-8 polypeptide of the present invention.

### SUMMARY OF THE INVENTION

In accordance with one aspect of the present invention, there is provided a mixture of novel polypeptides being truncated forms of human Ckβ-8 as well as biologically active and diagnostically or therapeutically useful fragments, analogs and derivatives thereof.

In accordance with another aspect of the present invention, there is provided isolated nucleic acid molecules encoding such polypeptides, including mRNAs, DNAs, cDNAs, genomic DNA as well as biologically active and diagnostically or therapeutically useful fragments, analogs and derivatives thereof.

In accordance with a further aspect of this invention, there is provided a process for producing such polypeptides by recombinant techniques which comprise culturing recombinant prokaryotic and/or eukaryotic host cells, containing nucleic acid sequences, under conditions promoting expression of said proteins and subsequent recovery of said proteins.

In accordance with yet a further aspect of the present invention, there is provided a process for utilizing such polypeptides, or polynucleotides encoding such polypeptides for therapeutic purposes, for example, to protect bone marrow stem cells from chemotherapeutic agents during chemotherapy, and to stimulate wound healing.

In accordance with another aspect of the present invention, there are provided antibodies against such a polypeptide.

In accordance with a further aspect of the present invention, there are provided antagonists to such a polypeptide, which may be used to inhibit the action of such a polypeptide, for example, to treat aplastic anemia, myelodysplastic syndrome, asthma and arthritis.

In accordance with another aspect of the present invention, there is also provided nucleic acid probes comprising nucleic acid molecules of sufficient length to specifically hybridize to the Ckβ-8 nucleic acid sequence.

In accordance with a further aspect of the present invention, there are provided diagnostic assays for detecting diseases related to the underexpression and overexpression of the polypeptide and for detecting mutations in the nucleic acid sequences encoding such a polypeptide.

In accordance with yet a further aspect of the present invention, there is a process for utilizing such a polypeptide, or polynucleotides encoding such a polypeptide, as research reagents for in vitro purposes related to scientific research, synthesis of DNA and manufacture of DNA vectors, for the purpose of developing therapeutics and diagnostics for the treatment of human disease.

These and other aspects of the present invention should be apparent to those skilled in the art from the teachings herein. Detailed Description of the Invention DNA sequences encoding polypeptides structurally related to the pro-inflammatory “intercrine” chemokine superfamily have been described. The polynucleotide sequence encoding full length Ckβ-8 (120 amino acids) was derived from an aortic endothelium cDNA library. The present invention concerns novel shortened forms of Ckβ-8 containing only the 82, 76, 75 or 74 C-terminal amino acids of the long form. The N-terminal amino acids as well as the 21 residue signal peptide sequence are cleaved. As with the long form of Ckβ-8, the first two cysteine residues in these clones are in adjacent positions placing them in the “C-C” or beta subfamily of chemokines. The presence of this motif distinguishes them from the a subfamily of chemokines (“CXC”) in which the first two cysteine residues are separated by one amino acid.

The polynucleotide sequences encoding the mature polypeptides (SEQ ID NOs: 1, 2, 3, and 4) may be represented in any nucleic acid form such as RNA, ssDNA, genonic DNA, etc. including additional coding and non-coding sequences associated with the polypeptide. Furthermore, due to the degeneracy of the amino acid code, any DNA coding sequence which encodes the same mature polypeptides shall be considered within the scope of this invention. Any variants of this sequence, allelic or non-naturally occurring shall also be included. The present invention also includes polynucleotides in which the coding sequence is fused in the same reading frame to another DNA sequence which aids in expression or secretion of the protein from a cell or serves as a marker to identify the protein in cells (e.g., HA tag).

This invention further relates to polynucleotide sequences which hybridize to the described sequence with at least 50% and preferably 70% sequence identity. These polynucleotides encode polypeptides which preferably retain the same biological function or activity as the mature polypeptide. Alternatively, the sequence may be polynucleotides which have, preferably, 50 bases that have identity to, and hybridize to the sequences of the present invention, but do not retain activity. Such sequences may be used as a probe or PCR primer.

The polypeptides of the present invention may be recombinant polypeptides, natural polypeptides, or synthetic polypeptides. Derivatives of these polypeptides may be one in which one or more amino acid substitutions are present; one in which one or more amino acids contain substituent groups; one in which the mature protein is fused with another compound; or one in which additional amino acids are fused to mature proteins.

The polypeptides of this invention are preferably provided in isolated form, and preferably purified to homogeneity.

The present invention also relates to vectors which include polynucleotides of this invention, host cells which are genetically engineered with this vector, and the production of polypeptides of the invention by recombinant techniques. Host cells are genetically engineered with the vectors of this invention (cloning or expression). The culture conditions for these cells in order to activate promoters, select transformants, or amplify the truncated Ckβ-8 gene will be apparent to those skilled in the art.

This polynucleotide sequence may be included in any one of a variety of expression vectors so long as they are replicable and viable in the host. The polynucleotide sequence inserted into the expression vector is under the control of an appropriate promoter to direct mRNA synthesis (e.g., LTR promoter). The expression vector also contains a ribosome binding site for translation initiation, a transcription terminator, enhancer elements which will increase transcription (e.g., SV40 late enhancer), and appropriate selectable markers to ensure maintenance of the vector in the host (e.g., ampicillin resistance gene). Host cells can be higher eukaryotic cells such as mammalian or insect cells, lower eukaryotic cells such as yeast, or prokaryotic cells such as bacteria, introduction of this construct into the host cell can be effected by a variety of protocols. All of the aforementioned steps will be obvious to those skilled in the art. Cloning steps as well as host and vector selection are well known in the art. (Sambrook et al., Molecular Cloning: A Laboratory Manual, Second Edition, Cold Spring Harbor, N.Y., (1989).

Proteins can be synthesized in the host cells under the control of an appropriate promoter. Cell-free translation systems can also be used to produce such proteins using RNAs derived from the DNA constructs of the present invention. Alternatively, the polypeptide of this invention can be synthetically produced by conventional peptide synthesizers.

Following transformation or transfection of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and purified to homogeneity by a variety of methods (e.g., chromatography, HPLC) Depending upon the host cell employed, the polypeptide may be modified in some manner (e.g., glycosylation). Polypeptides of the invention may also include an initial methionine residue.

The long form of Ckβ-8 is a chemoattractant for leukocytes and therefore may be employed in a variety of immunoregulatory and inflammatory functions as well as a number of disease conditions. This protein is expressed predominantly in tissues of hemopoietic origin. One important biological effect of the Ckβ-8 polypeptide on leukocytes is stimulation of the mobilization of Ca++ reserves. This mobilization pertains to functional activation of the cell. It has been found that stimulation of differentiated EOL-3 cells with a mixture containing four shortened forms of Ckβ-8 (SEQ ID NOS: 1-4) results in an immediate increase in intracellular Ca++ which is dose dependent. When tested individually, the short form having SEQ ID NO: 4 was the most active. In contrast, the long form of Ckβ-8 was approximately 1000 times less potent than the short forms at mobilizing Ca++. The potency of the short forms of the present invention may have implications for the effectiveness of therapeutic applications of this chemokine including, but not limited to, protection of bone marrow stem cells from chemotherapeutic agents during chemotherapy, removal of leukemic cells, stimulation of an immune response, regulation of hematopoiesis and lymphocyte trafficking, treatment of psoriasis, solid tumors, enhancing host defenses against resistant chronic and acute infection, and stimulation of wound healing.

The polynucleotides and polypeptides of the present invention may be used as a research reagents, in synthesis of DNA and manufacture of DNA vectors, and for the purpose of developing therapeutics and diagnostics for the treatment of human disease (e.g., in the expansion of immature hematopoietic progenitor cells). Fragments of this truncated Ckβ-8 polynucleotide sequence may also be used as a hybridization probe to isolate other genes which have a high sequence similarity. Techniques for these kinds of experiments are known to those skilled in the art.

This invention is also related to the use of these sequences as part of a diagnostic assay for detecting diseases or susceptibility to diseases related to the presence of mutations in the nucleic acid sequences. Such diseases are related to under-expression of the chemokine polypeptides. For example, individuals carrying mutations in the Ckβ-8 gene may be detected at the DNA level by a variety of techniques such as PCR. Genetic testing based on DNA sequence differences may be achieved by detection of alteration in electrophoretic mobility of DNA fragments in gels with or without denaturing agents. The detection of specific DNA sequence may be achieved by methods such as hybridization, RNase protection, chemical cleavage, direct DNA sequencing, RFLP analysis, and Southern blotting of genomic DNA. Mutations can also be detected by in situ analysis.

The present invention also relates to a diagnostic assay for detecting altered levels of Ckβ-8 protein in various tissues since an over-expression of the proteins compared to normal control tissue samples may detect the presence of a disease or susceptibility to a disease, for example, a tumor. Assays used to detect levels of Ckβ-8 protein in a sample derived from a host are well known to those of skill in the art and include radioimmunoassays, competitive-binding assays, Western blot analysis, ELISA assays and “sandwich” assays.

This invention provides a method for identification of the receptors for the chemokine polypeptides. The gene encoding the receptor can be identified by numerous methods known to those of skill in the art including ligand panning and FACS sorting.

An alternative approach for receptor identification involves photoaffinity linking the labeled polypeptides with the cell membrane or extract preparations that express the receptor molecule. The labeled complex can be isolated and subjected to protein microsequencing. The amino acid sequence obtained would be used to design a set of degenerate oligonucleotide probes to screen a cDNA library to identify the genes encoding the putative receptors.

This invention provides a method of screening compounds to identify agonists and antagonists to the chemokine polypeptide of the present invention. Chemotaxis may be assayed by placing cells chemoattracted by these polypeptides on top of a filter with pores large enough to admit the cells (5 mm). Solutions of potential agonists or antagonists are placed in a bottom chamber so that cells migrate or are prevented from migrating through the membrane over a period of time. Alternatively, the receptors of Ckβ-8 would be incubated with a labeled polypeptide in the presence of a compound. The ability of the compound to either block this interaction or interact with the receptor in the absence of polypeptide could be measured.

Examples of potential truncated Ckβ-8 antagonists include antibodies, oligonucleotides which bind to the polypeptides or polypeptides which bind to the receptor of the wild-type polypeptide, but fail to retain biological activity. Antisense technology used to control gene expression can also be a potential antagonist The availability of the aforementioned technologies should be obvious to those skilled in the art.

Antagonists may be employed to treat infectious diseases such as silicosis, sarcoidosis, idiopathic pulmonary fibrosis, idiopathic hyper-eosinophilic syndrome, and endotoxic shock—all by preventing production of the polypeptide of the present invention. The antagonists may also be employed for treating atherosclerosis, by preventing monocyte infiltration in the artery wall. Antagonists may be employed to treat histamine-mediated allergic reactions and various immunological disorders involving the dermis.

The antagonists may also be used to treat chronic and acute inflammation by preventing the attraction of monocytes to the wound area They may also be used to treat inflammatory pulmonary disease, general inflammation, and rheumatoid arthritis by preventing monocyte influx into affected areas.

Antagonists may also be employed to treat cases of bone marrow failure in aplastic anemia or myelodysplastic syndrome. They may also be used to treat asthma and allergy as well as subepithelial basement membrane fibrosis which is a feature of the asthmatic lung.

The chemokine polypeptide and agonists and antagonists may be employed in combination with a suitable pharmaceutical carrier such as saline, buffered saline, dextrose, water, glycerol, ethanol, and combinations thereof. The formulation should suit the mode of administration. The invention also, provides a pharmaceutical pack or kit comprising one or more containers filled with one or more of the ingredients of the pharmaceutical compositions of this invention. The polypeptides, agonists, and antagonists may be employed in conjunction with other therapeutic compounds.

The pharmaceutical compositions may be administered in a convenient manner such as by topical, intravenous, intraperitoneal, intramuscular, intratumor, subcutaneous, intranasal, or intradermal routes in an amount which is effective for treating the specific indication. These applications should be evident to those skilled in the art.

The chemokine polypeptides, agonists, or antagonists which are polypeptides, may be employed in accordance with the present invention by expression of such polypeptides in vivo. This type of gene therapy is well known in the art. For example, cells from a patient may be engineered with DNA or RNA-encoding the short form of Ckβ-8 polypeptide ex vivo and the engineered cells expressing these polypeptides provided to the patient. Similarly, cells may be engineered in vivo for expression of a polypeptide in vivo.

As known in the art, the use of a retroviral particle containing RNA encoding the polypeptide of the present invention may be used. Retroviral plasmid vectors are used to transduce packaging cell lines (e.g., PE501) by various means (e.g., electroporation) to form producer cell lines. In a preferred embodiment, the retroviral expression vector containing the polynucleotide sequences, is under the control of a promoter such as LTR and contains a selectable drug resistance marker (e.g., neo). The preparation of these vectors and resulting cell lines should be familiar to those skilled in the art and from the techniques contained herein. The producer cell line generates infectious retroviral vector particles which include the nucleic acid sequence(s) encoding the polypeptides. Such retroviral vector particles then may be employed to transduce eukaryotic cells, for example, fibroblasts or endothelial cells, either in vitro or in vivo. The transduced eukaryotic cells will then express the nucleic acid sequence(s) encoding the protein.

The sequences of the present invention are also valuable for chromosome identification. The sequences are specifically targeted to and can hybridize with a particular location on an individual chromosome. The mapping of DNA to chromosomes is an important step in correlating genes associated with disease. One technique known to those of skill in the art involves preparing primers used for PCR screening of somatic cell hybrids containing individual human chromosomes. Only those hybrids containing the human gene corresponding to the primer will yield an amplified fragment. Other mapping strategies known to those of skill in the art include, but are not limited to: in situ hybridization, prescreening with labeled flow-sorted chromosomes, sublocalization to fragments of specific chromosomes using the same PCR primers, and preselection by hybridization to construct chromosome specific-cDNA libraries. Fluorescence in situ hybridization (FISH) of cDNA clones to a metaphase chromosomal spread can be used to provide a precise chromosomal location in one step.

Once a sequence has been mapped to a precise chromosomal location, the physical position of the sequence can be correlated with genetic map data. The relationship between genes and disease can be identified through linkage analysis. Any differences in the cDNA or genomic sequences between affected and unaffected individuals can be indicative of disease. For example, a mutation in the DNA found only in the affected individuals may be the causative agent of the disease.

The polypeptides, their fragments or other derivatives, or analogs thereof, or cells expressing them can be used as an immunogen to produce antibodies thereto. These antibodies can be, for example, polyclonal or monoclonal antibodies. The present invention also includes chimeric, single chain and humanized antibodies, as well as Fab fragments, or the product of an Fab expression library. Various procedures known in the art may be used for the production of such antibodies and fragments. Polyclonal antibodies prepared by standard procedures can be used to isolate the polypeptides from tissue expressing that polypeptide. For preparation of monoclonal antibodies, any technique which provides antibodies produced by continuous cell lines cultures can be used. Techniques described for the production of single chain antibodies can be adapted to produce single chain antibodies to immunogenic polypeptides of this invention. Also, transgenic mice may be used to express humanized antibodies to immunogenic polypeptides of this invention.

The present invention will be further described with reference to the following examples; however, it is to be understood that the present invention is not limited to such examples. In order to facilitate understanding of the following examples certain frequently occurring methods and/or terms will be briefly described.

The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan. The various restriction enzymes used herein for “digestion” of DNA are commercially available and their reaction conditions, cofactors and other requirements were used as would be known to the ordinarily skilled artisan. Size separation of the cleaved fragments is performed using 8% polyacrylamide gel.

Oligonucleotides refers to either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated. Unless otherwise stated, transformation was performed as described in the method of Graham, F. and Van der Eb, A., Virology 1973, 52, 456-457.

The following examples are provided for illustrative purposes only and are rot intended to limit the invention.

### EXAMPLES

### Example 1

Bacterial Expression and Purification of Ckβ-8

The DNA sequence encoding Ckβ-8, ATCC# 75676 was initially amplified using PCR primers corresponding to the 5′, and 3′ end sequences of the processed Ckβ-8 protein (minus the signal peptide sequence) and the vector sequences 3′ to the Ckβ-8 gene. The 5′ oligonucleotide primer has the sequence 5′ TCAGGATCCGTCACAAAAGATGCAGA 3′ (SEQ ID NO: 5) and the 3′ primer has the sequence 5′CGCTCTAGAGTAAAACGACGGCCAGT 3′ (SEQ ID NO: 6). These primers respectively contain BamHI and XbaI restriction sites used for cloning into the polylinker region of the ampicillin resistant bacterial expression vector PQE-9 (Qiagen, Inc., Chatsworth, Calif.). The amplified sequences were ligated into PQE-9 in frame with the sequence encoding for the histidine tag and the ribosome binding site (RBS). This ligation was used to transform E. coli strain M15/rep 4 (Qiagen) which contains multiple copies of plasmid pREP4. pREP4 expresses the lacI repressor and also confers kanamycin resistance. Transformants were selected by their ability to grow on LB plates supplemented with ampicillin/kanamycin. Clones containing the desired constructs (isolated and confirmed by restriction analysis) were grown overnight in liquid culture in LB media supplemented with both Amp (100 μg/ml) and Kan (25 μg/ml). This culture was used to inoculate a large culture at a ratio of 1:100 to 1:250. The cells were grown to an optical density 600 of between 0.4 and 0.6. IPTG (isopropyl-B-D-thiogalacto-pyranoside) was then added to a final concentration of 1 mM. Cells were then grown an extra 3 to 4 hours, then harvested by centrifugation. The cell pellet was solubilized in the chaotropic agent 6 M Guanidine HCl, clarified, and subjected to chromatography on a Nickel-Chelate column under conditions that allow for tight binding by proteins containing the 6-His tag (Hochuli, E. et al. J. Chromatography 1984, 411, 177-184). Truncated Ckβ-8 (95% pure) is eluted from the column in 6 M guanidine HCl pH 5 and for the purposes of renaturation adjusted to 3 M guanidine HCl, 100 mM sodium phosphate, 10 mM glutathione (reduced) and 2 mM glutathione (oxidized). After incubation in this solution for 12 hours, the protein was dialyzed to 10 mM sodium phosphate.

### Example 2

Expression of recombinant Ckβ-8 in COS Cells

The expression of plasmid, CMV-Ckβ-8 HA is derived from a vector pcDNAI/Amp (Invitrogen) containing the ampicillin resistance gene and the CMV promoter followed by a polylinker region, SV40 intron, and polyadenylation site. A DNA fragment encoding the truncated Ckβ-8 sequences and an HA tag fused in frame to the 3′ end is cloned into the polylinker region of the vector; therefore, the recombinant protein expression is directed under the CMV promoter. The HA tag corresponds to an epitope derived from the influenza hemagglutinin protein as previously described (I. Wilson et al. Cell 1984, 37:767). The infusion of HA tag to the target protein allows easy detection of the recombinant protein with an antibody that recognizes the HA epitope.

For expression of the recombinant short forms of Ckβ-8. COS cells are transfected with the expression vector by DEAE—Dextran method (J. Sambrook, E. Fritsch, T. Maniatis, Molecular Cloning: A Laboratory Manual, Cold Spring Harbor Laboratory Press, (1989)). Cells are labeled for 8 hours with 35S- cysteine two days post transfection. Culture media are then collected and cells are lysed with. detergent (RIPA buffer (150 mM NaCl, 1% NP-40, 0.1% SDS, 1% NP40, 0.5% DOC, 50 mM Tris, pH 7.5) (Wilson, I. et al., ID. 1984,37:767). Both cell lysate and culture media are precipitated with a HA specific monoclonal antibody. Proteins precipitated are analyzed on 15% SDS-PAGE gels.

### Example 3

Expression and Purification of Short Form of Chemokine Ckβ-8 using a Baculovirus Expression System

SF9 cells were infected with a recombinant baculovirus designed to express the Ckβ-8 cDNA. Cells were infected in a 10 liter culture at an MOI of 2 and cultured under low serum conditions at 28° C. for 72-96 hours. Cellular debris from the infected culture was removed by low speed centrifugation. Protease inhibitor cocktail was added to the supernatant at a final concentration of 20 μg/ml (1 μg/ml leupeptin, 1 μg/ml E-64 and 1 mM EDTA). These particular culture conditions (i.e., low serum) produced several NH2-terminal truncated forms of the Ckβ-8 polypeptide. The level of the Ckβ-8 in the supernatant was monitored by loading 20-30 μl of supernatant on a 15% SDS-PAGE gel. The shortened forms of Ckβ-8 were detected as visible bands, corresponding to an expression level of several mg per liter. Truncated Ckβ-8 was further purified through a three-step purification procedure: 1) Heparin binding affinity chromatography. Supernatant of baculovirus culture was mixed with ⅓ volume of buffer containing 100 mM HEPES/MEM/NaOAc pH 6 and filtered through 0.22 μm membrane. The sample was then applied to a heparin binding column (HE1 poros 20, BIO-Perceptive System Inc.). The short forms of Ckβ-8 was eluted at approximately 300 mM NaCl in a linear gradient of 50 to 500 mM NaCl in 50 mM HEPES/MES/NaOAc at pH 6; 2) Cation exchange chromatography. The protein enriched from heparin chromatography was subjected to a 5-fold dilution with a buffer containing 50 mM HEPES/MES/NaOAc pH 6. The resultant mixture was then applied to a cation exchange column (S/M poros 20, BIO-Perceptive System Inc.). Truncated Ckβ-8 was eluted at 250 mM NaCl in a linear gradient of 25 to 300 mM NaCl in 50 mM HEPES/MES/NaOAc at pH 6; 3) Size exclusion chromatography. Following the cation exchange chromatography, truncated Ckβ-8 was further purified by applying to a size exclusion column (HW50, TOSO HAAS, 1.4×45 cm.). Amino Acid sequencing of the purified sample showed a mixture of at least four dominant sequences corresponding to the four truncated forms of the protein (SEQ ID NOs: 1, 2, 3, and 4).

### Example 4

Stimulation of EOL-3 Cells and PBLs with a Mixture Comprising Short Forms of Ckβ-8

EOL-3 cells were grown under standard growth conditions and differentiated for two weeks with 1 μM Na Butyrate. PBLs (peripheral blood leukocytes) were obtained from human donors by vena puncture. PBLs were purified by standard techniques. Both cell preparations were separately loaded with FURA-2 (1 μM) for 45 minutes. Cells at 1×106/ml (2 ml total) were transferred to plastic cuvettes. The fluorescent spectrophotometer was adjusted to give a baseline recording before the short forms (871a, 871b. 871c, 871RP) or long form (889) of Ckβ-8 was added at a concentration of 0.33 to 33 nM. The increase in fluorescence was monitored with the spectrophotometer. The change in free Ca++ inside the cell was calculated by first lysing the cells with Triton ×100 in the presence of excess Ca++ (to obtain Fmax) and then adding 5 mM EGTA, to obtain Fmin. The ratio of free Ca++ can be calculated from the min and max values. MCP-1 (monocyte chemotactic protein-1) is a known chemokine and was used as a positive control.

What is claimed is:

1. A polypeptide comprising SEQ ID NO: 1.

2. A polypeptide comprising SEQ ID NO: 2.

3. A polypeptide comprising SEQ ID NO: 3.

4. A polypeptide comprising SEQ ID NO: 4.

5. A composition comprising a mixture of the polypeptides of claims 1, 2, 3, and 4.

6. A vector comprising a polynucleotide encoding polypeptide of claim 1, 2, 3, or 4.

7. A host cell genetically engineered with a vector of claim 6.

8. A process for producing a polypeptide of claim 1, 2, 3, or 4 comprising expressing the polypeptide from a host cell genetically engineered with a vector comprising a polynucleotide encoding the polypeptide of claim 1, 2, 3, or 4.

9. An agonist for the polypeptide of claim 1, 2, 3 or 4.

10. An antagonist for the polypeptide of claim 1, 2, 3, or 4.

11. A method for the treatment of a patient having a need of Ckβ-8 comprising administering to a patient an effective amount of the polypeptide of claim 1, 2, 3, or 4.

12. A method of treatment of a patient having a need of Ckβ-8 comprising administering to a patient an effective amount of the composition of claim 5.

13. A method for the treatment of a patient having a need to inhibit Ckβ-8 comprising administering to a patient an effective amount of an antagonist of claim 10.

14. A method for identifying antagonists and agonists of a polypeptide of claim 1, 2, 3, or 4 comprising combining selected test cells, a compound to be screened and a polypeptide of claim 1, 2, 3, or 4; and determining if the compound is an effective agonist or antagonist based upon the response of the test cells.

15. A process for diagnosing a disease or a susceptibility to a disease related to the underexpression of a polypeptide of claim 1, 2, 3 or 4 comprising determining if there is a mutation in the nucleic acid sequence encoding the polypeptide.


SEQUENCE LISTING (10 sequences)

SEQ ID NO: 1 - protein, 82 aa, Homo sapiens
ENPVLLDRFHATSADCCISYTPRSIPCSLLESYFETNSECSKPGVIFLTKKGRRFCANPSDKQVQVCMRMLKLDTRIKTRKN

SEQ ID NO: 2 - protein, 77 aa, Homo sapiens
LDRFHATSADCCISYTPRSIPCSLLESYFETNSECSKPGVIFLTKKGRRFCANPSDKQVQVCMRMLKLDTRIKTRKN

SEQ ID NO: 3 - protein, 76 aa, Homo sapiens
DRFHATSADCCISYTPRSIPCSLLESYFETNSECSKPGVIFLTKKGRRFCANPSDKQVQVCMRMLKLDTRIKTRKN

SEQ ID NO: 4 - protein, 75 aa, Homo sapiens
RFHATSADCCISYTPRSIPCSLLESYFETNSECSKPGVIFLTKKGRRFCANPSDKQVQVCMRMLKLDTRIKTRKN

SEQ ID NO: 5 - DNA, 26 bp, Oligonucleotide Primer
tcaggatccgtcacaaaagatgcaga

SEQ ID NO: 6 - DNA, 26 bp, Oligonucleotide Primer
cgctctagagtaaaacgacggccagt

SEQ ID NO: 7 - protein, 83 aa, Homo sapiens
MENPVLLDRFHATSADCCISYTPRSIPCSLLESYFETNSECSKPGVIFLTKKGRRFCANPSDKQVQVCMRMLKLDTRIKTRKN

SEQ ID NO: 8 - protein, 78 aa, Homo sapiens
MLDRFHATSADCCISYTPRSIPCSLLESYFETNSECSKPGVIFLTKKGRRFCANPSDKQVQVCMRMLKLDTRIKTRKN

SEQ ID NO: 9 - protein, 77 aa, Homo sapiens
MDRFHATSADCCISYTPRSIPCSLLESYFETNSECSKPGVIFLTKKGRRFCANPSDKQVQVCMRMLKLDTRIKTRKN

SEQ ID NO: 10 - protein, 76 aa, Homo sapiens
MRFHATSADCCISYTPRSIPCSLLESYFETNSECSKPGVIFLTKKGRRFCANPSDKQVQVCMRMLKLDTRIKTRKN